(19) United States
(12) Patent Application Publication
(10) Pub. No.: US 2023/0288342 A1
(43) Pub. Date: Sep. 14, 2023

(54) HYDROGEN GAS SENSORS USING MAGNETO-PLASMONIC NANOLATTICES

(71) Applicants: BATTELLE SAVANNAH RIVER ALLIANCE, LLC, AIKEN, SC (US); UNIVERSITY OF GEORGIA RESEARCH FOUNDATION, INC., ATHENS, GA (US)

(72) Inventors: GEORGE K. LARSEN, III, AIKEN, SC (US); THO D. NGUYEN, DACULA, GA (US); MINH THIEN PHAM, FALLS CHURCH, VA (US); HOANG M. LUONG, ATHENS, GA (US)

(21) Appl. No.: 18/105,071

(22) Filed: Feb. 2, 2023

Related U.S. Application Data
(60) Provisional application No. 63/318,489, filed on Mar. 10, 2022.

(51) Int. Cl. G01N 21/77; G01N 33/00

(52) U.S. Cl. CPC G01N 21/77; G01N 33/005; G01N 21/554

(57) ABSTRACT

Hydrogen gas sensors with low cost, fast response time, large detection range, high sensitivity, and excellent limit of detection are described. The devices include a composite formed on a nano-scale that both absorbs hydrogen and exhibits magneto-optical effects. Sensor readout is based on magneto-optical effects, which also may be enhanced through plasmonic coupling. The hydrogen sensors are fast and sensitive, as well as resistant to surface poisoning from common contaminants, such as carbon monoxide.

### CROSS REFERENCE TO RELATED APPLICATION

This application claims filing benefit of U.S. Provisional Patent Application Ser. No. 63/318,489 having a filing date of Mar. 10, 2022, which is incorporated herein by reference for all purposes.

### FEDERAL RESEARCH STATEMENT

This invention was made with Government support under Contract No. 89303321CEM000080, awarded by the U.S. Department of Energy. The Government has certain rights in the invention.

### BACKGROUND

Hydrogen (H₂) gas has the potential to be a dominant future energy earner, due to its high gravimetric energy density, sustainability, and lack of carbon emissions upon consumption. As hydrogen generation and hydrogen fuel cell technology continue to develop, the demand for hydrogen sensors for safely handling hydrogen gas in all stages of a hydrogen economy including production, distribution, storage, and utilization will also continue to rise. For hydrogen leakage detection and concentration controls, it is essential that hydrogen sensors have good stability, high sensitivity, rapid response time, and most importantly be “spark-free.” Ideally, hydrogen sensor performance targets specify a response time of 1 s at room temperature across a concentration range from 0.1% to 10% (see Table 1, below, which summarizes performance targets for stationary and automotive hydrogen sensors)—a performance goal that has been achieved by only a few sensors reported to date.

TABLE 1

| | Stationary | Automotive |
|---|---|---|
| Measuring range | Up to 4 vol. % H₂ (survive 100%) | |
| Lower detection limit | <0.1 vol. % | |
| Response time (t₉₀) | <30 s | <1s |
| Recovery time (t₁₀) | <30 s | <1s |
| Accuracy | ±10 % | ±5% |
| Ambient temperature | −20 to +50° C. | −40 to +125° C. |
| Ambient pressure | 80110 kPa | 62107 kPa |
| Ambient relative humidity | 20-80% | 0-100% |

| | |
|---|---|
| Lifetime | >5 years |

High performance hydrogen sensors are of importance not only in a future hydrogen economy but also needed in existing developed industries including the chemical industry for e.g., monitoring the hydrogen content in the fertilizer industry and refinement of crude oils in the petrochemical industry; the food industry for e.g., detecting the hydrogen gas as an end product or byproduct of a biological process, and leak detection; the medical industry for e.g., measurement of the hydrogen content in human breath to diagnose certain conditions such as lactose intolerance or bacterial overgrowth in intestines; and the environmental pollution control industry; among others.

Palladium (Pd) nanoparticles have been widely used in optical nano-plasmonic hydrogen sensors due to intrinsic hydrogen selectivity and sizable optical change upon absorption. However, Pd optical sensors have drawbacks such as hysteresis behavior and long response times, especially at plateau pressures.

Composite nanoparticles for hydrogen detection (e.g., those including a Pd composite) have been reported to be capable of either reducing the response time down to 1 s at 0.1% H₂ or achieving detection in a wide range of 1 ppm to 100% H₂. While such composite sensors have shown improvement in the art, no hydrogen sensor has been reported that can achieve both feats, i.e., fast response at low hydrogen content. For instance, the sensitivity of reported composite materials are reduced considerably in comparison to pure Pd counterparts. Moreover, composite, e.g., alloy, nanostructures can require more complicated and expensive fabrication processes.

Additionally, sample measurement and data processing using known hydrogen sensor materials are complicated, with expensive measurement setups that require a high resolution spectrometer to collect the transmission/extinction spectra, and complex data analysis that requires fitting process to determine the peak content location.

While improvements in the art has been made, room for further improvement exists.

### SUMMARY

According to one embodiment, disclosed is a hydrogen sensor that includes a substrate and a sensing layer on the substrate having a thickness of from about 2 nanometers to about 30 nanometers. The layer includes a composite including a magnetic material and a hydride-forming metal. The composite includes the magnetic material and the hydride-forming metal in a molar ratio of from about 1:1 to about 1:6.

Also disclosed is a hydrogen sensing system that includes a sensor that includes a substrate and a layer on the substrate having a thickness of from about 2 nanometers to about 30 nanometers. The layer includes a composite including a magnetic material and a hydride-forming metal. The composite includes the magnetic material and the hydride-forming metal in a molar ratio of from about 1:1 to about 1:6. The system also includes a source configured to impinge the sensor with a probing energy beam. In addition, the system includes a magnet, and the sensor is retained within a magnetic field of the magnet. The system also includes an analysis system configured to detect and analyze a resulting energy beam following impingement of the probing energy beam with the sensor.

Also disclosed is a method for detecting hydrogen. The method includes contacting a sensor retained within a magnetic field with a sample comprising hydrogen. The sensor includes a substrate and a layer on the substrate having a thickness of from about 2 nanometers to about 30 nanometers. The layer including a composite including a magnetic material and a hydride-forming metal. The composite includes the magnetic material and the hydride-forming metal in a molar ratio of from about 1:1 to about 1:6. The method also includes impinging the sensor with a probing energy beam and analyzing a magneto-optical response of a resulting energy beam following contact of the gas, the sensor, and the probing energy beam, the analysis providing information regarding the presence or quantity of the hydrogen in the sample

### BRIEF DESCRIPTION OF THE FIGURES

A full and enabling disclosure of the present subject matter, including the best mode thereof to one of ordinary skill in the art, is set forth more particularly in the remainder of the specification, including reference to the accompanying figures in which:

FIG. 1 illustrates a schematic of one embodiment of a hydrogen sensor fabrication process and a sensor as may be formed according to the process.

FIG. 2 illustrates an exemplary nanoparticle of a sensor including layered polymer coatings over a sensing layer.

FIG. 3A illustrates an exemplary nanoparticle of a sensor including a multilayer sensing layer and plasmonic layer structure.

FIG. 3B illustrates an exemplary nanoparticle of a sensor including a single sensing/plasmonic composite layer.

FIG. 4A illustrates nano-hole arrays of one embodiment of a sensor.

FIG. 4B illustrates bilayer plasmonic nano-lattices of one embodiment of a sensor.

FIG. 4C illustrates nanotriangle arrays of one embodiment of a sensor.

FIG. 4D illustrates nano-fans of one embodiment of a sensor.

FIG. 4E illustrates helically stacked sensing/plasmonic layers of one embodiment of a sensor.

FIG. 4F illustrates chiral nano-hole arrays of one embodiment of a sensor.

FIG. 4G illustrates helical nano-sized structures of one embodiment of a sensor.

FIG. 5 illustrates a schematic of three embodiments of multi-layer fabrication processes as may be utilized in forming sensors as disclosed herein.

FIG. 6 illustrates a magnetic-circular dichroism (MCD) measurement setup as may be utilized with disclosed sensors.

FIG. 7A illustrates an examination set-up for a sensor as described utilizing a transmissive MCD measurement technique.

FIG. 7B illustrates an examination set-up for a sensor as described utilizing a reflective MCD measurement technique.

FIG. 7C illustrates an examination set-up for a sensor as described utilizing a Faraday Rotation (FR) measurement setup.

FIG. 7D illustrates an examination set-up for a sensor as described utilizing a magneto-optical Kerr rotation (MOKE) measurement setup.

FIG. 8A illustrates hysteresis loops of nanoparticle-based sensors as disclosed herein measured under several H₂ pressures and vacuum (including data both before and after exposure to H₂).

FIG. 8B illustrates a ΔMCD=MCD (P_{H2}=0 mbar)−MCD (P_{H2}=1000 mbar) hysteresis loop including maxima changes of MCD signal at ±≈300 G.

FIG. 9A illustrates MCD hysteresis absorption and desorption isotherms of nanoparticle-based sensors as described herein. Arrows denote the sorption direction.

FIG. 9B illustrates response times of a nanoparticle-based hydrogen sensor to varying H₂ pressure pulses.

FIG. 9C presents ΔMCD response to stepwise decreasing H₂ pressure in the 0.75-5000 μbar range, measured at 1 Hz sampling frequency in a vacuum chamber.

FIG. 9D illustrates measured ΔMCD as a function of H₂ pressure derived from FIG. 3C. All experiments were performed at 30° C.

FIG. 10A provides a top-view SEM image of a packed array of nanoparticles of a sensor as described herein.

FIG. 10B provides an ultra-high-resolution SEM micrograph showing morphology of a nanoparticle sample of the array of FIG. 8A. Inset image provides grain size analysis for the area (denoted by a white-border rectangle) on the top of a nanoparticle.

FIG. 10C provides energy-dispersive X-ray spectroscopy (EDS) elemental maps of nanoparticle samples of the array of FIG. 8A. All scale bars in FIG. 8A-8C correspond to 200 nm.

FIG. 11A provides MCD spectra (MCD=CD(−2600 G)−CD(0 G)), and ΔMCD=MCD_{1000 mbar}−MCD_{0 mbar}, where CD(B), is the CD of a Pd₆₇Co₃₃ NP sensor measured at magnetic field B, and MCD_{1000 mbar} and MCD_{0 mbar} of the sensor, are measured at P_{H2}=1000 mbar and <0.01 mbar, respectively.

FIG. 11B presents MCD hysteresis loops of the sensor of FIG. 9A taken at λ=450 nm, measured under several H₂ pressures and vacuum (before and after exposed to H₂). Arrows denote the magnetization direction.

FIG. 11C presents ΔMCD hysteresis loops of nanoparticle (solid lines) and thin film (dashed lines) samples showing maxima changes of MCD signal at ˜±380 G and ˜±420 G, respectively. The thicker arrows indicate ΔMCD maxima to have a non-specific readout signal. The thinner arrows denote the magnetization direction.

FIG. 12A presents MCD spectra MCD_{1000 mbar} and MCD_{0 mbar} of a control PdCo thin film sample, measured at P_{H}_{₂}=1000 mbar and <0.01 mbar, respectively, and ΔMCD=MCD_{1000 mbar}−MCD_{0 mbar}.

FIG. 12B presents MCD hysteresis loops of a control PdCo thin film sample at λ=450 nm, measured under several H₂ pressures and vacuum (before and after exposed to H₂). Arrows denote the magnetization direction.

FIG. 12C a ΔMCD hysteresis loop of a control PdCo thin film sample showing maxima changes of MCD. Black arrows denote the magnetization direction.

FIG. 13A presents magneto-optical (MO) H₂ sorption isotherm of nanoparticles extracted at ΔMCD maxima (at +380 G). Inset: Sensor accuracy at specific normalized ΔMCD readout over H₂ pressure range of 101 μbar to 106 μbar.

FIG. 13B presents ΔMCD response of a sensor to stepwise decreasing hydrogen pressure in the 5000-4.6 μbar range, measured at 1 Hz sampling frequency in a vacuum chamber. Shaded areas denote the periods where the sensor was exposed to hydrogen.

FIG. 13C presents the ΔMCD response of a sensor (1 Hz of sampling frequency) with different hydrogen concentration (C_{H2}), measured in flowing nitrogen (400 ml/min). Shaded areas denote the periods where the sensor was exposed to H₂.

FIG. 13D presents measured ΔMCD response as a function of H₂ pressure/concentration derived from the data of FIG. 10B and FIG. 10C. The solid line denotes the defined LOD at 36=0.12 mdeg.

FIG. 13E presents raw absorption kinetic response of a sensor (with desorption pressure of <0.08 mbar) to varying H₂ pressure from 1000 to 1 mbar at 23° C. (32 Hz of sampling frequency).

FIG. 13F presents raw desorption kinetic response of a sensor (with desorption pressure of <0.08 mbar) to varying H₂ pressure from 1000 to 1 mbar at 23° C. (32 Hz of sampling frequency).

FIG. 13G presents extracted absorption time (t₉₀) and desorption time (t₁₀), derived from the data of FIG. 13E and FIG. 13F.

FIG. 14A provides a schematic illustrating a single-layer NP sensor and a triple-layer NP sensor as disclosed herein.

FIG. 14B presents ΔMCD response of a triple-layer NP sensor to stepwise decreasing hydrogen pressure in the 5000-4.6 μbar range, measured at 1 Hz sampling frequency in a vacuum chamber. Shaded areas denote the periods where the sensor was exposed to hydrogen.

FIG. 14C presents ΔMCD response of a triple-layer sensor (1 Hz of sampling frequency) with different hydrogen concentration (C_{H2}), measured in flowing nitrogen (400 ml/min). Shaded areas denote the periods where the sensor is exposed to hydrogen.

FIG. 14D presents measured ΔMCD response as a function of hydrogen pressure/concentration derived from data of FIG. 11B and FIG. 11C. The solid line denotes the defined LOD at 36=0.12 mdeg. Inset: a magnified view at low P_{H2}/C_{H2}.

FIG. 15A presents MCD hysteresis loops of a single-layer, double-layer, and triple-layer sensor, measured at λ=450 nm and P_{H2}=1000 mbar.

FIG. 15B presents raw absorption kinetics response of single-layer, double-layer, and triple-layer sensors to a 10 mbar H₂ pressure step, showing similar response times of t₉₀<0.5 s.

FIG. 16A presents MO hydrogen sorption isotherm of a sensor including nanoparticle (NP)/Teflon® AF 2400 (TAF) stacks extracted at ΔMCD maxima.

FIG. 16B presents ΔMCD response of a NP/TAF sensor to stepwise decreasing hydrogen pressure in the 5000-4.6 μbar range, measured at 1 Hz sampling frequency in a vacuum chamber. Shaded areas denote the periods where the sensor was exposed to hydrogen.

FIG. 16C presents ΔMCD response of a NP/TAF sensor (1 Hz of sampling frequency) with different hydrogen concentration (C_{H2}), measured in flowing nitrogen (400 ml/min). Shaded areas denote the periods where the sensor is exposed to hydrogen.

FIG. 16D presents measured ΔMCD response as a function of hydrogen pressure/concentration derived from data of FIG. 12B and FIG. 12C. The solid line denotes the defined LOD at 3σ=0.12 mdeg.

FIG. 16E presents raw absorption kinetics response (with desorption pressure of <0.08 mbar) of to varying H₂ pressure from 1000 to 1 mbar at 23° C. (32 Hz of sampling frequency).

FIG. 16F presents raw desorption kinetic response (with desorption pressure of <0.08 mbar) of to varying H₂ pressure from 1000 to 1 mbar at 23° C. (32 Hz of sampling frequency).

FIG. 16G presents extracted absorption time (t₉₀) and desorption time (t₁₀), derived from data of FIG. 15E and FIG. 15F.

FIG. 17A presents MO hydrogen sorption isotherm of a sensor including multiple polymeric coating layers extracted at ΔMCD maxima.

FIG. 17B presents the ΔMCD response of the sensor to stepwise decreasing hydrogen pressure in the 5000-4.6 μbar range, measured at 1 Hz sampling frequency in a vacuum chamber. Shaded areas denote the periods where the sensor was exposed to hydrogen.

FIG. 17C presents the ΔMCD response of the sensor (1 Hz of sampling frequency) with different hydrogen concentration (C_{H2}), measured in flowing nitrogen (400 ml/min). Shaded areas denote the periods where the sensor was exposed to hydrogen.

FIG. 17D presents the measured ΔMCD response as a function of hydrogen pressure/concentration derived from FIG. 17B and FIG. 17C.

FIG. 17E presents raw absorption kinetics response to varying H₂ pressure from 1000 to 1 mbar at 23° C. (16 Hz of sampling frequency).

FIG. 17F presents raw desorption kinetic response (with desorption pressure of <0.08 mbar) to varying H₂ pressure from 1000 to 1 mbar at 23° C. (16 Hz of sampling frequency).

FIG. 18A presents extracted absorption time (t₉₀) and desorption time (t₁₀) of a NP/TAF/PMMA sensor.

FIG. 18B presents ΔMCD response of a NP/TAF/PMMA sensor upon 100 cycles of 2% H₂ in synthetic gas (400 ml/min).

FIG. 18C presents time-resolved ΔMCD response of a NP/TAF/PMMA sensor to 12 pulses of 2% H₂ (top row), 3 pulses of 2% H₂ followed by 9 pulses of 2% H₂+5% CO₂ (2^{nd} row), 3 pulses of 2% H₂ followed by 9 pulses of 5% CH₄ (3^{rd} row), and 3 pulses of 2% H₂ followed by 9 pulses of 0.2% CO (bottom row).

FIG. 18D presents the sensor signal normalized to the one obtained with 2% H₂ in synthetic gas flow. The error bars denote the standard deviation from 9 cycles. The shaded areas denote the ±20% deviation limit from the normalized ΔMCD response with 2% H₂.

FIG. 18E presents time-resolved ΔMCD response of a NP/TAF/PMMA sensor to 10 pulses of 2% H₂ with different relative humidity (RH) of 0, 40%, and 90%.

FIG. 18F presents the signal normalized to the one obtained with 2% H₂ in dry condition. All measurements were performed at 23° C., using synthetic gas as carrier gas. The shaded areas denote the ±20% deviation limit from the normalized ΔMCD response with 2% H₂.

### DETAILED DESCRIPTION

Reference will now be made in detail to various embodiments of the disclosed subject matter, one or more examples of which are set forth below. Each embodiment is provided by way of explanation of the subject matter, not limitation thereof. In fact, it will be apparent to those skilled in the art that various modifications and variations may be made in the present disclosure without departing from the scope or spirit of the subject matter. For instance, features illustrated or described as part of one embodiment, may be used in another embodiment to yield a still further embodiment.

In general, disclosed are hydrogen sensors that can be formed at low cost while exhibiting fast response time, large detection range, high sensitivity, and excellent limit of detection. Disclosed sensors include a plurality of nano-sized structures (e.g., nano-hole arrays, nano-particle arrays, or combination of structures), that include both a magnetic material and a hydride-forming metal in a thin-layered composite sensing layer in which the magnetic material is present at a relatively high concentration.

The hydrogen sensors can provide excellent response using magneto-optical effects (e.g., Faraday rotation, Kerr rotation, reflective and/or transmissive magnetic-circular dichroism) of the nanoparticles to the presence of hydrogen due to surface plasmon resonance of the hydride-forming metal(s) included in close association with the relatively large amount of the magnetic material in the nanostructures. In disclosed systems, the interactions between hydrogen and the hydride forming metal(s) via the resonantly enhanced light/matter interaction due to surface plasmon resonances have been advantageously utilized. Practical application of these phenomena has led to the development of disclosed hydrogen sensors that can exhibit numerous advantages over previously known hydrogen sensors, including simple and multiple output readings, fast response time, and high sensitivity.

The working principle of a hydrogen sensor as disclosed herein relies on the fact that the conductance and optical permittivity of a metal changes as the metal sorbs hydrogen and undergoes a phase transformation into a metal hydride. Changes in conductance and optical permittivity are expressed through optical responses that can be resonantly captured and correlated with hydrogen pressure and hydrogen to metal ratio (H/M). Disclosed systems detect these changes by use of magneto-optical sensing platforms based on surface plasmon resonance concepts. Specific aspects of sensing systems utilized with disclosed sensors can be categorized by how the optical responses are induced, e.g., through localized surface plasmon resonance (LSPR), such as in a format that includes a plurality of single nanoparticles, e.g., nano-spheres, nano-disks, nano-triangles, nano-bipyramids, nano-wires, or any other single nano-particle shape or combination thereof; or by propagating surface plasmon polariton (SPP), such as in nano-hole arrays or prism/grating couplings.

One of the most critical drawbacks of previously known hydrogen sensors is hysteretic behavior. Beneficially, in one embodiment disclosed hydrogen sensors can be hysteresis free. Another drawback of previously known hydrogen sensors is that the sorption kinetics are slow due to the structural change of the sorbent material. This structural change leads to large equilibrium response times for the sensors, especially when the pressure is in the plateau regime where the phase transition occurs. Disclosed sensors, in contrast, can exhibit a fast response time. For instance, disclosed sensors can obtain a response time of about 1 s at room temperature across a hydrogen concentration range from about 0.1 volume % to about 10 volume %.

In addition to other beneficial aspects, disclosed sensors can obtain a performance level of about 1 ppm to 100% volume concentration (or about 1 μbar to about 1000 mbar) of hydrogen and can be highly selective for hydrogen with an abundant sensitivity to allow for accuracy of about ±5% or better.

It should be appreciated that, although the present subject matter will generally be described herein in terms of a magneto-optical analysis system and the use of magneto-optical measurement methods, various other analysis systems and related measurement methods may be utilized in accordance with aspects of the present subject matter. For instance, in addition to magneto-optics (e.g., including Faraday rotation, MCD, MOKE, etc.), various other suitable optical techniques may include, but are not limited to, polarized or unpolarized transmission/reflection measurement techniques, scattering measurement techniques, resistance-based measurement techniques, and/or the like.

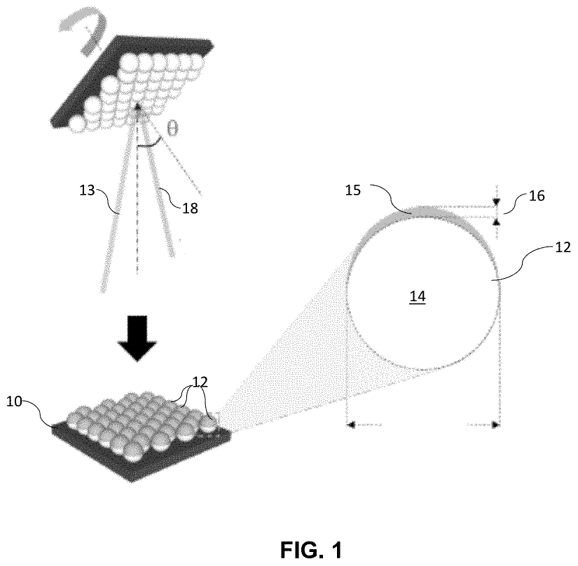

FIG. 1 illustrates one embodiment of a sensor 10 that includes a plurality of individual nano-sized structures 12 in an array. Each structure 12 includes a substrate 14 and a sensing layer 15 that forms a continuous coating over at least a portion of the substrate, i.e., the sensing layer 15 is not in the form of a plurality of separated islands, but includes a continuous layer that is continuous across about 5% or more of the surface area of the substrate 14 that carries the layer 15. As indicated, the sensing layer 15 need not cover the entire surface of the substrate 14. However, a continuous sensing layer 15 can cover about 5% or more of the surface area of the substrate 14, such as about 15% or more, or about 25% or more, or about 30% or more, or about 35% or more, or about 40% or more in some embodiments. The coating formed by the sensing layer 15 may, in various embodiments, have any suitable shape, including being circular, triangular, fan-shaped, and/or the like.

The sensing layer 15 includes a composite that incorporates a hydride-forming metal and a magnetic material. The hydride-forming metal can include, by way of example and without limitation, palladium, platinum, magnesium, titanium, vanadium, etc., as well as any alloy thereof. The magnetic material can include any magnetic material or combination thereof that can be provided in a composite with the hydride-forming metal. By way of example, the magnetic material can include a ferromagnetic material that can include one or more of cobalt, iron, nickel, gadolinium, etc. The number of components in the composite material of a sensing layer 15 can include one or more hydride-forming metals as well as one or more magnetic materials.

Due to slow hydrogen diffusion coefficient in a hydride-forming metal composite, the nano-size features of the sensors can increase the rate of hydrogen sorption/desorption in the sensing layer 15, which can lead to fast response times for the sensors. For instance, at the thickest point(s) 16 of the sensing layer 15, the sensing layer 15 can define a thickness of about 2 nanometers or greater, such as from about 2 nanometers to about 30 nanometers, such as from about 5 nanometers to about 20 nanometers in some embodiments. Moreover, it will be understood that a sensing layer 15 need not have a single thickness across the entirety of the sensing layer 15. For instance, when considering a substrate 14 that defines a curvature at the surface that carries the sensing layer 15 as illustrated in FIG. 1, the sensing layer 15 can vary in thickness across the sensing layer 15, depending primarily on the method used to form the sensing layer 15. For instance, curvature of a substrate surface that carries the sensing layer 15 can increase the surface-to-volume ratio of a structure 12 as compared to a two-dimensional particle (e.g., a disc), which can further improve response time of a sensor. In any case, a sensing layer 15 can be continuous over at least a portion of the surface area of the substrate 14 and can be about 2 nanometers or greater at the thickest point(s) 16.

The relative proportion of the magnetic and hydride-forming metal can be utilized to ensure that the sensing layer 15 possesses a sufficiently large magnetic property in conjunction with fast hydrogen sorption kinetics that, upon hydrogen sorption, can substantially change a magnetic property of the sensing layer 15. For instance, magnetic material(s) and hydride-forming metal(s) of a sensing layer 15 can be present in the layer in a molar ratio of from about 1:1 to about 1:6, such as from about 1:1 to about 1:4, such from about 1:1 to about 1:3, such as from about 1:1 to about 1:2 in some embodiments.

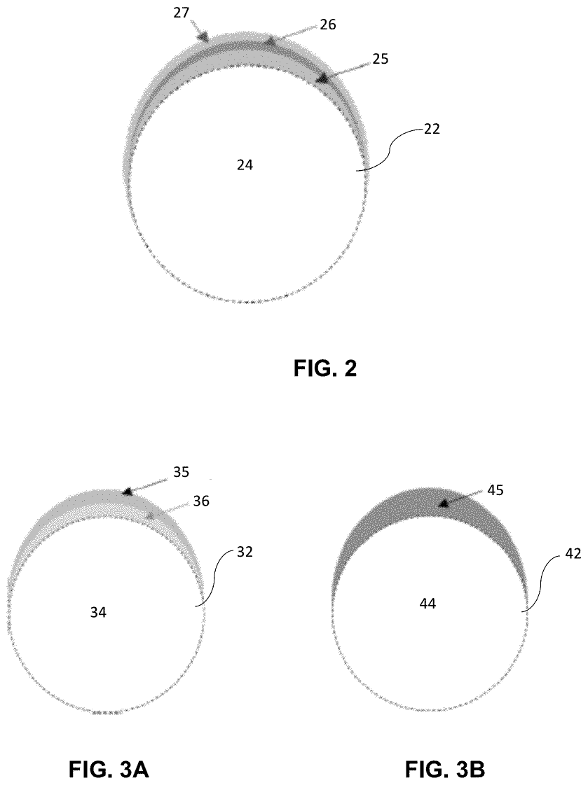

FIG. 2 illustrates one embodiment of a nano-sized structure 22 that can be incorporated in a sensor. As illustrated, the structure 22 can include a substrate 24 and a sensing layer 25 on the substrate 24 that includes a composite including a hydride-forming material and a magnetic material. In this embodiment, the structure 22 further includes a layer 26 and a layer 27 that overcoats the sensing layer 25. The additional layers 26, 27 can include materials that can enhance function of the sensor. For instance, in one embodiment one or both of an overcoat layer 26, 27 can include a polymeric layer that can allow specific gas penetration and/or reduce the activation barrier of hydrogen sorption/desorption. Such features can enable gas sensing selectivity and significantly reduce the response time of a sensor.

By way of example, and without limitation, a coating layer 26 and/or 27 can include a polymer that can allow selective gas penetration and in particular can allow penetration of hydrogen while avoiding sensor deactivation by one or more compounds that could poison the sensor (e.g. CO, CO, NH₄, NO₂, etc.) and thereby can prolong the lifetime of sensor. Long-term exposure of a sensor to poisonous gas species that can exist in trace levels in ambient air (e.g., CO) can significantly degrade the performance of a sensor, which can reduce sensitivity, accuracy, and slow down the sensor response, and thereby shorten the lifetime of the sensor. Selective enhancement of a sensor through inclusion of one or more coating layers 26, 27 can also be utilized to reduce the activation barrier of hydrogen absorption/desorption, which can reduce the response time of a sensor.

Polymeric materials as may be utilized in forming a coating layer can include, without limitation, polytetrafluoroethylene (PTFE) (e.g., Teflon®), polymethyl-methacrylate (PMMA), polyimides (PI), polysulfones (PS), siloxanes such as polydimethylsiloxane (PDMS), and the like that can allow hydrogen penetration to reach a composite sensing material. Teflon® is a perfluorinated polymer as PTFE and can be desirable in some embodiments as it possesses high chemical resistances and hydrophobicity, while having a high gas permeability and low refractive index, which are favorable for forming a sensing system.

In one embodiment, a nano-sized structure 22 can include multiple coating layers, as illustrated in FIG. 2. For instance, a first coating layer 26 can be formed of a polymeric material that can reduce the activation barrier of hydrogen sorption/desorption, e.g., a PTFE, and a second coating layer 27 can be formed of a material that can allow selective gas penetration, e.g., a PMMA. Of course, a sensor structure need not include multiple coating layers, and any combination of enhancement layers and materials is encompassed herein.

In one embodiment, a sensor can include a noble metal additive, which can provide a plasmonic enhancement response that can strongly enhance magneto-optical signals of a sensor. By way of example, a nano-sized structure can include gold, silver, platinum, etc., or any combination thereof in a nano-sized structure so as to enhance plasmonic coupling. As illustrated in FIG. 3A, a nano-sized structure 32 can include a noble metal incorporated into a separate layer 36 adjacent a sensing layer 35 that includes the composite. In another embodiment, illustrated in FIG. 3B, a noble metal can be incorporated in a sensing layer 45 that also includes the hydride-forming metal and the magnetic material composite. A sensor that includes a noble metal, either in a single composite layer or in a separate layer from the sensing layer can also include one or more additional layers, e.g., one or more polymeric layers as discussed previously. In some embodiments, addition of a noble metal and plasmonic coupling enabled thereby can significantly boost the signal magnitude as well as sensitivity of a sensor.

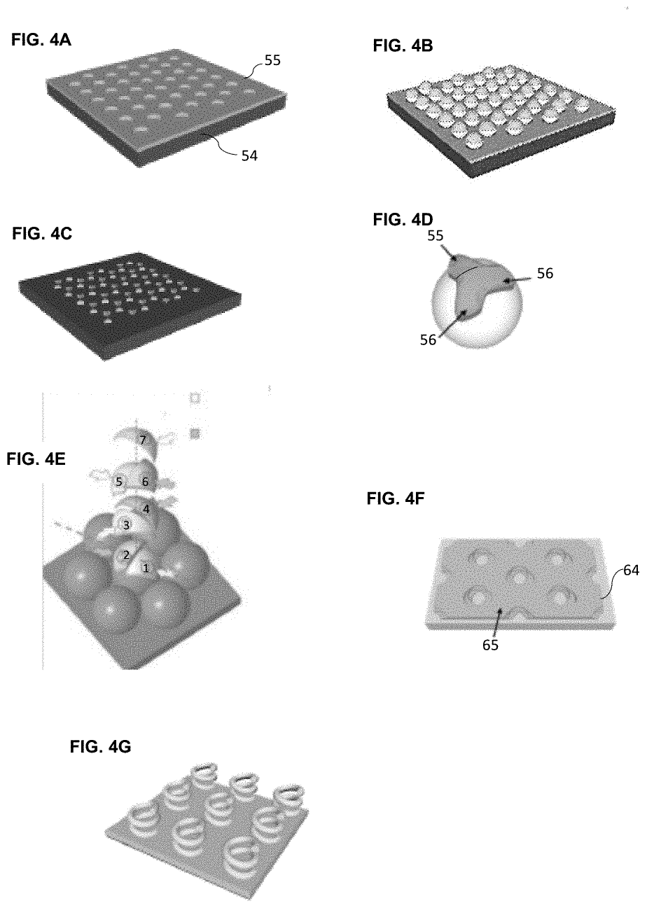

A substrate 14, 24, 34, 44 that can carry a sensing layer 15, 25, 35, 45 can be provided on a nano-sized scale. For instance, as illustrated in FIG. 1, a substrate 14 can be in the form of a bead having a cross-sectional dimension on a nanometer scale, e.g., about 300 nanometers or less, such as from about 50 nanometers to about 300 nanometers, or from about 100 nanometers to about 200 nanometers in some embodiments. A substrate in the form of a bead can include a generally spherical bead, as in FIG. 1, or a bead of any other suitable shape, e.g., triangular, square, polygonal, etc. Moreover, a sensor can include nanostructures that are achiral (such as nano-hole arrays, nano-triangle arrays, bilayer plasmonic nano-lattices, etc. (e.g., as illustrated in FIG. 4A, FIG. 4B, FIG. 4C), or can include a chiral structure (such as nano-fan, helically stacked layers, chiral nano-hole arrays, helical nanostructures, etc. (e.g., as illustrated in FIG. 4D, FIG. 4E, FIG. 4F, or FIG. 4F). By way of example, FIG. 4D illustrates a sensor that includes a composite sensing layer 55 and a plasmonic enhancement layer (e.g., a noble metal-containing layer) 56 in adjacent areas on a substrate, and optionally also overlaying one another in at least a portion of the area covered by the sensing layer 55. In the embodiment of FIG. 4E, an exploded image of a nanostructure is provided in which the substrate 1 can include a plurality of layers 2, 3, 4, 5, 6, 7 in a stacked arrangement, with each layer overlapping an underlayer only partially, providing a helically stacked arrangement. At least one of the layers 2-7 can be a sensing layer, with other layers of the structure providing some beneficial feature, e.g., a noble metal, a protective coating, etc. In some embodiments, the shape of the substrate can be selected so as to enhance the magneto-optical signal of the sensor. For instance, a chiral structure as in FIG. 4D, FIG. 4E, FIG. 4F, and FIG. 4G can couple and enhance the circular dichroism and the magnetic circular dichroism signal of a sensor.

In some embodiments, a supporting substrate can be formed of a material that can allow hydrogen penetration. For instance, a substrate 14, 24, 34, 44 in the form of a bead as in FIG. 1-3 or a planar substrate 54, 64 that supports a thin nano-hole array sensing layer 55, 65, can be formed of a material that can allow hydrogen penetration for improved access of hydrogen to the sensing layer, e.g., polystyrene, PMMA, etc.

The nanostructures of a hydrogen sensor can be fabricated using standard micro and nanofabrication techniques. In one embodiment, illustrated in FIG. 1, a combination of electron beam co-evaporation and glancing angle deposition (GLAD) techniques can be used for deposition of a hydride-forming material 13 and a magnetic material 18 together in a single sensing layer 15. A GLAD co-evaporation deposition method can be desirable in some embodiments, as it can allow for mass production of hydrogen sensors with very high reproductivity. It is also a cost-effective, and time-effective formation method.

Of course, other tem plating techniques as are known in the art are encompassed herein, and the method for formation of a sensing layer or other layers of a sensor are not particularly limited. By way of example, tem plating techniques such as direct laser writing or e-beam lithography may be used in formation of the nanostructures of a sensor.

Enhancing the sensor signal as well as improving the limit of detection (LOD) without sacrificing a fast response time is challenging, as it requires a higher volume of active materials (e.g. thicker sensing layers, or higher surface coverage) which is normally associated with a lower surface to volume ratio (slower sorption kinetics) or necessitates of changing sensor nanoarchitecture. In one embodiment, to further improve the function, a hydrogen sensor can include a plurality of layers, each of which including a plurality of nanostructures that include sensing layers thereon.

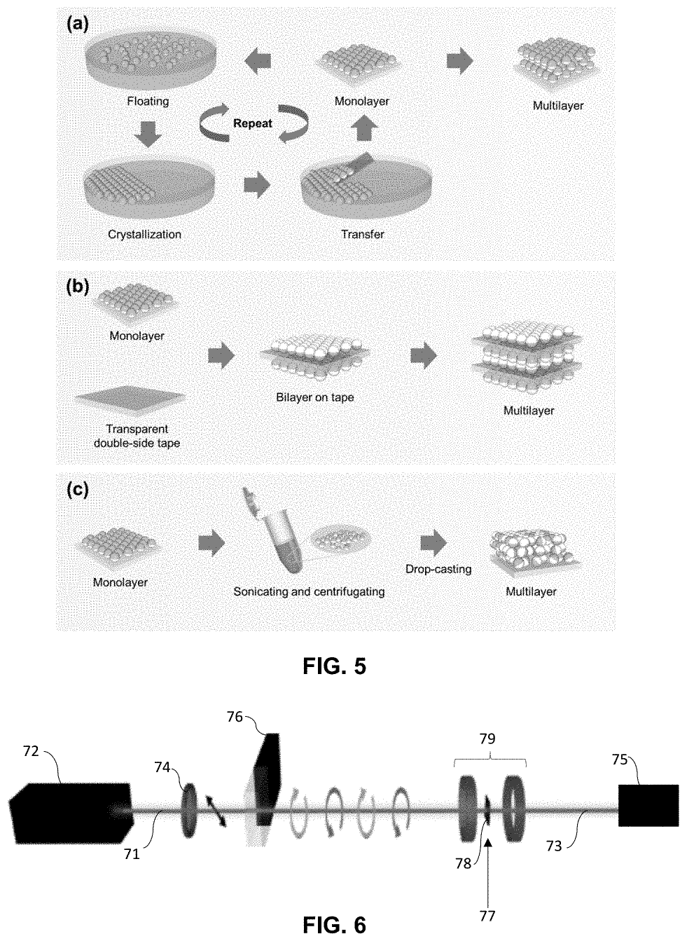

FIG. 5 illustrates several such multilayer structures as well as exemplary methods for forming a multi-layer sensor. By way of example and without limitation, a multilayer sensor can be formed in various embodiments by transferring monolayers to other substrates by using a water-floating (FIG. 5 at (a)), thorough utilization of an adhesive tape (FIG. 5 at (b)), by sonicating and recasting (FIG. 5 at (c)), or alternatively by simply stacking multiple individual sensors on top of each other.

Through control and arrangement of the nanostructures of each stacked layer, hydrogen molecules can easily and rapidly penetrate through a stacked nanostructure arrangement and reach the sorption sites on sensing layers. This along with the high surface to volume ratio of the nanostructures can provide fast response characteristic of disclosed sensors. Moreover, through increase of the volume of sensing materials by increased number of stacked layers, n, a sensor can provide an n-fold enhancement in sensor signal and push the limit of detection for a sensor to a lower hydrogen pressure to concentration ratio (P_{H2}/C_{H2}).

Rather than using conventional optical response analysis techniques for hydrogen sensing systems that incorporate disclosed nanostructures, the presence of the magnetic material in the sensing layers in a relatively high concentration allows for utilization of magneto-optical (MO) signal readout (e.g. MO Faraday rotation (FR), MO Kerr rotation (MOKE), reflective/transmissive magnetic-circular dichroism (MCD), etc. as well as combinations thereof) in disclosed systems. Upon exposure to hydrogen gas, the magnetic property of the thin sensing layers of a system can be monotonically quenched causing a large change in the MO signal, the intensity of which can be used as an efficient readout mechanism. The MO readout can provide a superior approach as compared to conventional optical readout systems as MO-based sensing can have enhanced sensitivity (%/mbar) by an order of magnitude in comparison to that of conventional optical detection. Moreover, the MO response can be less sensitive to stray light and refractive index of the ambient environment.

One embodiment of a system as disclosed herein is illustrated in FIG. 6. As illustrated, a system can include a source 72 for a probing energy beam, e.g., a diode laser or the like, that can emit a probing energy beam 71. A wavelength for a probing energy beam 71 can be any wavelength suitable for the particular MO analysis technique utilized by the system. In one embodiment the probing wavelength can be any wavelength. In embodiments, a desirable wavelength for a probing energy beam 71 can be chosen to optimize maximum sensor signal-to-noise (SNR) ratio, as well as the sensor price and power consumption.

In one embodiment, a sensing system can include a polarizer 74 and a phase sensitive photo-elastic modulator 76 for modulation of the probing energy beam 71. Incorporation of one or more optical modulation devices in a system can be utilized to reduce noise in the system and allow a LOD as low as several hundreds of part-per-billion (ppb) hydrogen concentration detection. However, modulation of the probing energy beam 71 is not a requirement, and in other embodiments, the probing energy beam 71 need not be polarized or otherwise modulated. In particular, the probing energy beam 71 can be any suitable type of signal suitable for an MO system, such as, and without limitation to, MOKE/FR, reflective MCD, etc., with a preferred signal depending on the nature of the particular sensor 78 of a system.

Following any desired modulation, the probing energy beam 71 can be impinged upon a sensor 78 as disclosed herein. The sensor 78 can generally be located in fluid communication with a sample 77 that can contain hydrogen.

A system can also include a magnet 79 in conjunction with the sensor 78 such that the sensor is retained within the magnetic field of the magnet 79. The magnet 79 can be a permanent magnet or an electromagnet, as desired. Following contact of the probing energy beam 71 with the sensor 78 and the sample 77, a resulting energy beam 73 can be directed to an analysis system 75 that can include one or more components, e.g., a differentiate photodiode, analyzer, photodiode, etc. Beneficially, disclosed systems can provide high accuracy and fast results while using a relatively small magnetic field. For instance, a magnetic field of a system can be about 300 G or less. Though, of course, large magnetic fields are also encompassed herein.

The analysis device and system 75 can be configured to analyze the intensity of the resulting energy beam 73 following contact of the probing energy beam 71 with the sensor 78, rather than spectrum used in the conventional sensor. Beneficially, the working principle of the MO-based sensor is simpler and therefore potentially has a lower cost of production as compared to other previously known systems.

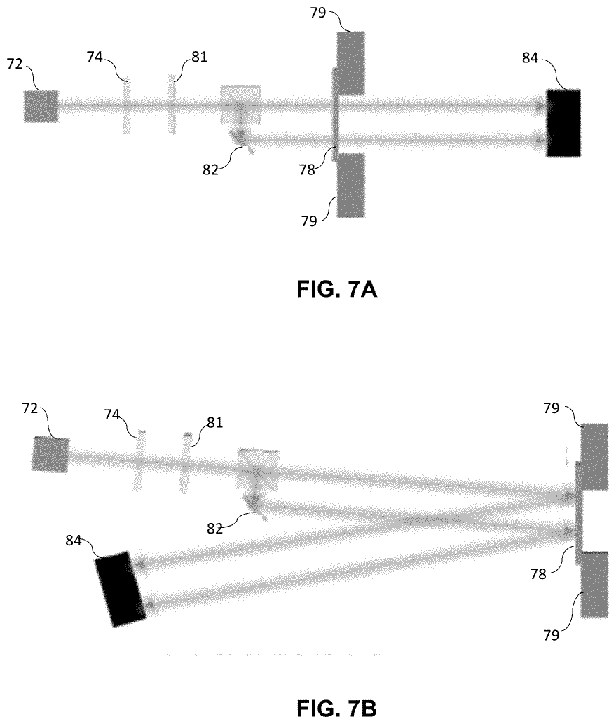

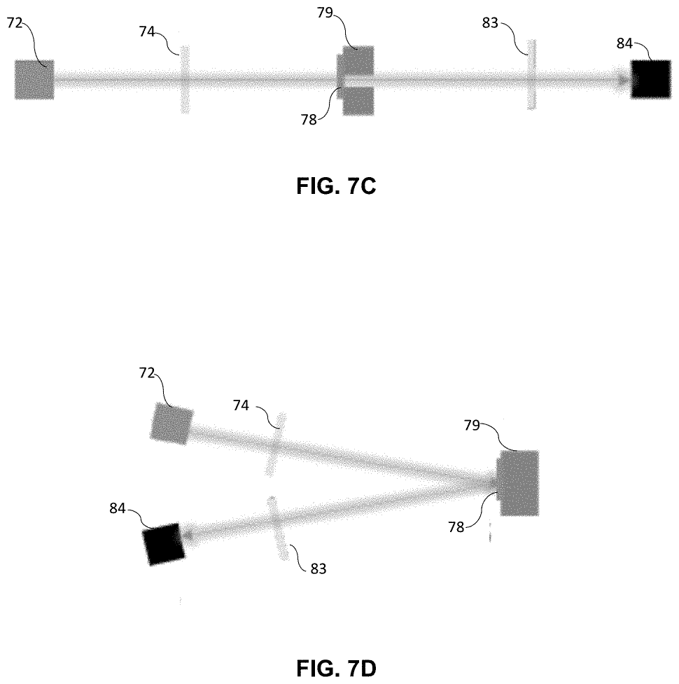

FIG. 7A-FIG. 7D illustrate several representative embodiments of sensing systems as disclosed herein based on using an MCD/FR/MOKE analysis technique. As illustrated, a setup can include inexpensive optics such as a diode laser 72, one or more polarizers 74, quarter-wave plate 81 (as in FIG. 7A, FIG. 7B), a photodetector 84 and one or more magnets 79. In the systems of FIG. 7C and FIG. 7D polarizer 74 and analyzer 83 have been placed in extinction positions (i.e., polarization axes are perpendicular) and the signal at a photodetector 84 would be approximately zero. Any changes in optical rotation (for MOKE and FR rotation) or different intensity between left and right circular polarization (for MCD) absorption induced by the interaction of hydrogen with a sensor 78 can be detected by the intensity change at the photodetector 84, which can be correlated to the hydrogen concentration without any complex fitting process in comparison to optical spectra readout. In embodiments, components can be further integrated into a photonic circuit to reduce the size and cost.

Disclosed hydrogen sensor systems can satisfy rigorous requirements for stationary as well as automotive application. For instance, disclosed systems can reach an ultra-low limit of detection of about 500 parts per billion, and can thus provide for detection of the hydrogen concentration in the air. Such capabilities can allow for improved applications, e.g., monitoring environment pollution.

Among all of the performance target for automotive H₂ safety sensors released by the US DOE, the sensor response time target of t₉₀<1 second (t₉₀, the time required to reach 90% of the final equilibrium response) is one of the most crucial and challenging targets to achieve. Indeed, only a few optical H₂ sensors have explicitly demonstrated this capability at P_{H2}≤1 mbar (or CH₂≤0.1% V) and room temperature. Disclosed sensors can meet or exceed such requirements, and in particular can bring the response time t₉₀ to under the 1 second benchmark, as well as to minimize the recovery time t₁₀ (the time required to reach 10% of the signal with releasing pressure of P_{H2}≤0.08 mbar).

The present invention may be better understood with reference to the examples, set forth below.

### Example 1

Deionized water was used for all experiments. polystyrene nanospheres (Polysciences Inc., 200 nm diameter), deionized water (18 MΩ cm), and ethanol (Sigma-Aldrich, 98%) were used to create nanosphere monolayers. Palladium (99.95%) and cobalt (99.95%) from Kurt. J Lesker Company was utilized for e-beam depositions.

A hexagonal close-packed nanosphere monolayer was prepared according to an air/water interface method and used as a template for electron beam deposition. The substrate beads were coated with Pd and Co vapors simultaneously under a constant deposition rate of 0.05 nm/s, and the sample holder rotated azimuthally with a constant rotation rate of 30 rpm during deposition process. The deposited thickness and rate were monitored by two independent quartz crystal microbalances (QCM), and the composition of the film could be adjusted by controlling the deposition rates of Pd and Co. Total calibrated quartz crystal monitor (QCM) thickness was 15 nm, and the Pd₈₅Co₁₅ atomic composition of the film was calculated from the deposition thicknesses t_{Co} and t_{Pd} for Pd and Co.

The MCD of the sensor with different H₂ pressure (P_{H2}) was characterized. The samples were loaded into a vacuum chamber with quartz optical windows, and P_{H2} was controlled by two pressure transducers (PX409-USBH, Omega). Before any measurements, the chamber and samples was flushed with >10 times with hydrogen/nitrogen cycles. All the measurements were performed at 30° C.

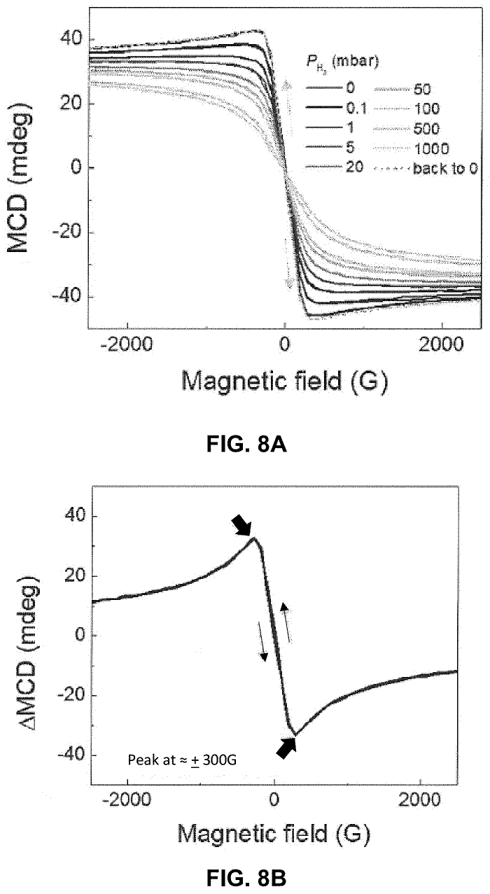

The MCD hysteresis loops of the sensor with different P_{H2} are shown in FIG. 8A (measured at wavelength λ=450 nm). All the curves showed zero coercivity field implying that the particles were superparamagnetic. The hysteresis loop before and after hydrogenation/dehydrogenation exposure was reversible and reproducible. FIG. 8B illustrates the changes of MCD signal (when the sensor was under vacuum and 1000 mbar of H₂) at different applied external magnetic fields. The maxima change of MCD signal (ΔMCD) was at relatively low magnetic field of ±300 G (arrows). Therefore, for hydrogen sensing, the external magnetic field was set at 300 G for all experiments.

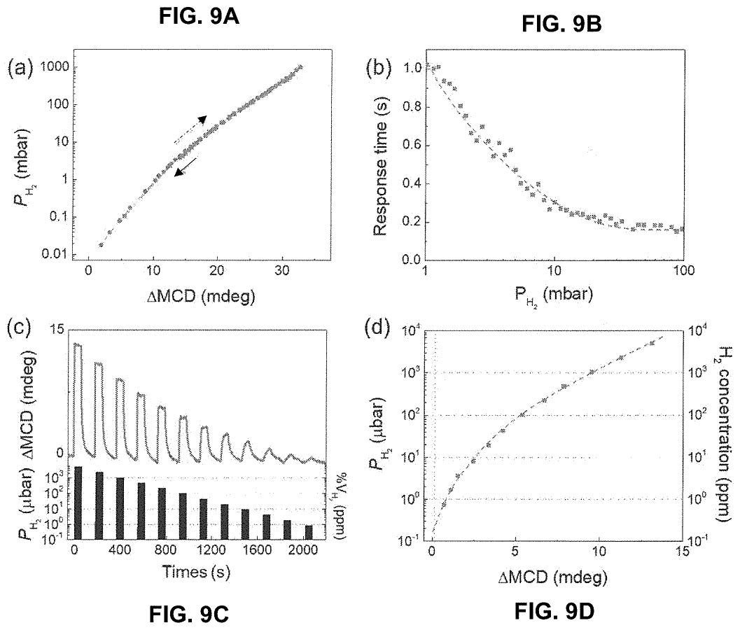

MCD hydrogen absorption and desorption isotherm at 30° C. for the sensor was examined. FIG. 9A shows a “hysteresis free” characteristic curve, with a wide detection range from <0.1 mbar (0.01% V) to 1000 mbar (100% V) of H₂. Most remarkably, the response times of the sensor were below 1 s over the range of 1-1000 mbar (0.1-100% V) (FIG. 9B). In addition, the response time at P_{H2}=40 mbar (or 4% ¾V, which is lower ignition concentration point of H₂) was approximately 0.16 s.

The limit of detection of the sensor was derived by exposure to pulses of gradually decreasing H₂ pressures in vacuum (from 5000 to 0.75 μbar, the lowest pressure attainable in the setup) and by measuring ΔMCD at 1 Hz sampling rate (FIG. 9C). The defined limit of detection was 36=0.12 mdeg, where 0 was the noise of the acquired signal (i.e. 0.040 mdeg, data not shown). The limit of detection of the sensor of approximately 0.2 μbar in pure hydrogen was extrapolated (which is equivalent to concentration of 200 ppb, FIG. 9D). This is an order of magnitude lower than any optical hydrogen sensor ever reported.

### Example 2

Polystyrene beads (Polysciences Inc., D=200 nm and 500 nm), ethanol (Sigma-Aldrich, 98%) and deionized water (18 MΩ cm) were used for monolayer formation. Palladium (99.95%), silver (99.99%), and cobalt (99.95%) from Kurt. J Lesker Company were utilized for electron beam depositions. Teflon® AF 2400 (Dupont) (TAF) and PMMA (Sigma Aldrich, Mw=15000) were used for polymer coating.

A monolayer of polystyrene nanosphere (D=200 nm and 500 nm in two different runs) on glass substrate was prepared by the air/water interface method. The monolayer substrate was used as a template for GLAD, where Pd and Co were electron beam co-evaporated to form the sensors. The vapor incident angle was set at θ=50°. The total deposition thickness was t_{dep}=15.0 nm and the vapor rates of Pd and Co were independently controlled to achieve a film composition of Pd₆₇Co₃₃. A thin film sample was deposited on a glass substrate simultaneously, which served as a control sample.

For TAF coating, the TAF powder was thermally evaporated to form a uniform coating with a thickness of about 30 nm. For PMMA coating, PMMA powder was dissolved in acetone at a concentration of 10 mg/ml, which was stirred and heated at 80° C. for 5 minutes and then cooled down to room temperature to fully dissolve the PMMA. The solution was the spin-coated on the particle sample at a speed of 5000 rpm for 120 s. The final thickness of the PMMA film was about 100 nm.

The MCD signal was collected by using a setup based on phase sensitive photo-elastic modulator (PEM) technique, similar to that illustrated in FIG. 6. A Xenon lamp and monochromator were used as a light source to obtain MCD spectra. For other measurements such as isotherms, time responses and limit of detection (LOD) measurements, a 50-mW blue diode laser at 450 nm (CNI Optoelectronics Technology, TEM-F-450) was used to replace the Xenon lamp and monochromator as a light source to provide a well collimated light and a better signal to noise ratio. For measurements in vacuum mode, the sensor was stored in a vacuum chamber with two quartz windows. The hydrogen pressure was monitored by three independent pressure transducers which cover the pressure range of 10⁻⁶ to 1.1 bar (two PX409-USBH, Omega and a Baratron, MKS). The time response and LOD measurements were performed at the sampling frequencies of 32 Hz ( 1/512 s integration time with 16 averages) and 1 Hz ( 1/512 s integration time with 512 averages), respectively, using a high-sampling rate lock-in amplifier (SR830 DSP Lock-In Amplifier, Stanford Research Systems). For measurements in flow mode, ultra-high purity hydrogen gas (Airgas) was diluted with ultra-high purity nitrogen gas (Airgas) or synthetic gas (Airgas) to targeted concentrations by commercial gas blenders (GB-103, MCQ Instruments). The gas flow rate through the sensor was kept constant at 400 ml/min for all measurements. All experiments were performed at constant 23° C.

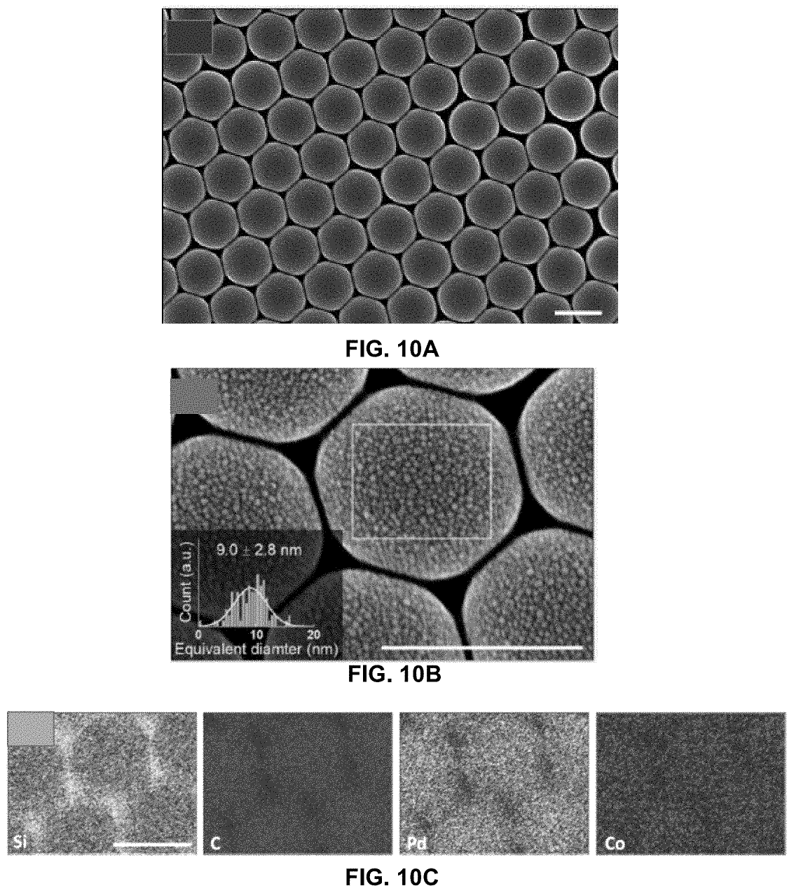

The structures of the sensor included a Pd₆₇Co₃₃ composite hemispherical sensing layer with a nominal thickness of 15 nm on top. The sensor included the structures in a hexagonal closed-packed nanosphere monolayer, which was verified by scanning electron microscopy (SEM) and energy-dispersive spectroscopy (EDS) elemental mapping. Using ultra-high-resolution SEM (SU-9000, Hitachi), the morphology of the sensor included of many sub-10-nm granules (FIG. 10A and FIG. 10B). These granules aggregated and fully covered the top surface of the polystyrene spheres and had an average equivalent diameter of 9.0±2.8 nm (FIG. 10B, inset), which is consistent with the observations of film morphologies presented in previous work. In addition, EDS elemental mapping showed a uniform distribution of Pd and Co on the top of each nanosphere (FIG. 10C) and confirmed a ratio of Pd to Co of 64:36, which is in agreement with the 67:33 ratio obtained by controlling deposition rate and thickness. The bead size, D=200 nm was chosen to ensure that the uniform hexagonal closed-packed nanosphere monolayer could be fabricated with a large surface-to-volume ratio in the magnetic caps for enhancing the hydrogen sorption kinetics.

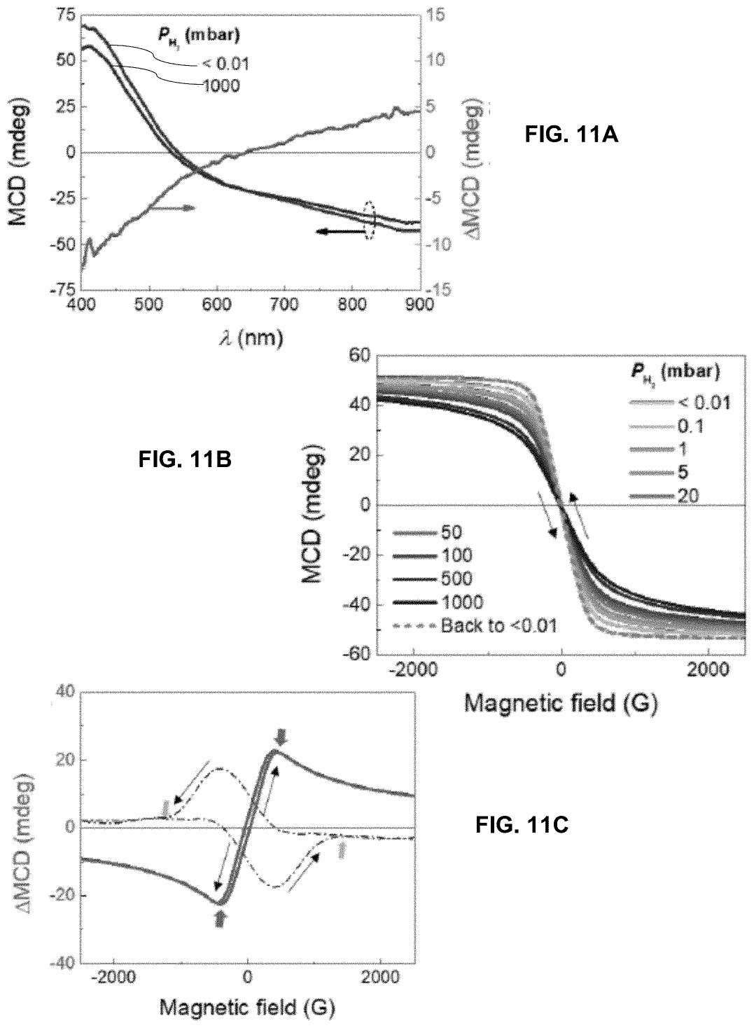

MCD was chosen as a H₂ sensing readout signal, since the MCD of a glass substrate would be negligible at an external magnetic field of B<3000 G. More importantly, the possible curvature-induced magnetochiral effect structures could cause superior MCD kinetics under hydrogenation that is suitable for the sensing readout signal. FIG. 11A depicts the MCD spectra of a sensor taken at B=−2600 G, where the magnetization was essentially saturated as seen in FIG. 11B. The MCD signal sharply decreased when the wavelength λ increased from 400 nm and approached zero at λ_{zero}≈535 nm. The sign of the MCD flipped when λ>λ_{zero}, There existed a kink near λₖᵢₙₖ≈600 nm from which the slope of MCD versus λ was noticeably smaller at λ>λₖᵢₙₖ.

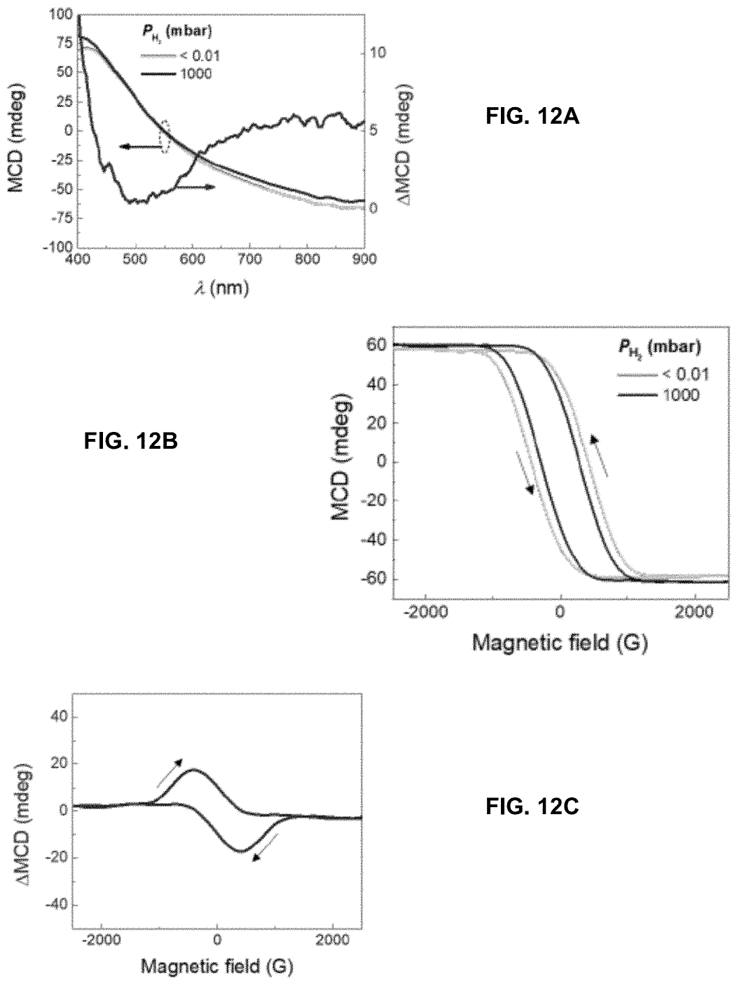

Upon hydrogenation (FIG. 11A), the MCD magnitude of the sensor slightly decreased, and the slope of the MCD versus λ above and below λₖᵢₙₖ became smaller. The change of the MCD signal, ΔMCD=MCD_{1000 mbar}−MCD_{0 mbar}, where MCD_{1000 mbar}, MCD_{0 mbar} are the MCD signal when P_{H2}=1000 and <0.01 mbar, monotonically increased with the probe wavelength from −12.6 to +4.6 mdeg, while the ΔMCD in a control PdCo thin film sample was always positive and fluctuated between +12.4 to 0 mdeg (FIG. 12A-FIG. 12C). It was noted that the MCDs of the glass substrate and a stand-alone nanosphere monolayer did not change upon their exposure to hydrogen.

FIG. 11B shows the MCD magnetization curve of the sensor at λ=450 nm as the external magnetic field was swept between B=−2600 G and +2600 G. At a very low P_{H2}<0.01 mbar or when no H₂ was present, the MCD hysteresis loop exhibited a typical soft magnetic ferromagnetic characteristic with relatively small coercivity field (Hc<10 G). In addition, the hysteresis curve showed an out-of-plane hard-axis behavior with almost no remanence, and saturation field was at a relatively low magnetic field (˜600 G). The unique MCD property in the magnetic caps was in contrast to the MCD hysteresis of the control PdCo thin film (at λ=450 nm, FIG. 12A-FIG. 12C) that shows an out-of-plane easy-axis behavior with relatively large remanence, large Hc (>400 G), and high saturation field (>1000 G). The behavioral differences between MCD hysteresis loops of the curved nanostructures and the thin film can be attributed to several factors: (i) the curvature of the nanostructures is likely to yield the alteration of film thickness/shape that induces an additional effective energy contribution (geometrical potential) to the total energy of the magnetic sensing layer; (ii) morphologically, the sensor includes numerous separated and loosely bound nanostructures in hexagonal-lattice arrays with almost-zero/negligible magnetic interactions between individual particles, while in the PdCo thin film, the continuity of the film supports the domain formation and domain wall shifting, especially when hydrogen is present; (iii) the simultaneous deposition of Pd and Co on a curved substrate leads to formation of grains with tilted growth direction and lateral grain sizes (FIG. 10B), which might significantly influence the magnetic properties of the sensors.

The introduction of hydrogen instantly rendered a reduction in MCD magnitude regardless of external magnetic field strength, and the magnitude of the changes (ΔMCD) increased when P_{H2} increases (FIG. 11B). In addition, a decreasing absolute slope value in MCD curve (from −500 G to +500 G) versus magnetic field was observed that caused the saturation field shifting to a higher field strength. This implies a reduction in out-of-plane component upon hydrogenation. 30 At P_{H2}=1000 mbar and B˜2500 G, the MCD signal still does not reach a fully-saturation, while the remanence and coercivity remain small and relatively the same as without hydrogen. More importantly, a reversible MCD signal was observed upon dehydrogenation (FIG. 11B).

FIG. 11C presents ΔMCD of a sensor (solid loop) at different B, which is the signal difference between P_{H2}=1000 mbar curve and P_{H2}<0.01 mbar curve in FIG. 11B. Two ΔMCD maxima were found at relatively low B of ±380 G (FIG. 11C, large arrows). Remarkably, a narrow hysteresis in ΔMCD magnetization loop which could instigate non-specific sensor readout could be seen at only B<380 G, however no hysteresis with relatively large ΔMCD was observed at B=380 G (˜22.5 mdeg) or stronger magnetic field. A constant B was therefore set at −380 G or +380 G for all sensing characterizations of the sensor, in order to obtain the highest signal to noise ratio leading to high sensor accuracy. In contrast, ΔMCD of the thin film depicted in FIG. 11C (dash loop) and FIG. 12C, showed non-specific sensing readout at maximal ΔMCD at ˜400 G field, and specific readout at ±1300 G but with much smaller ΔMCD of ˜2.5 mdeg. Consequently, the superior magnetic property of the magnetic sensing layer enhances the sensitivity by an order of magnitude while using much smaller magnetic field. It is worth noting that this low magnetic field strength can be generated by using a low-cost commercial permanent magnet, which significantly simplifies the sensor assembly since no high-power electromagnet is required, making this sensing platform lightweight and cost-efficient.

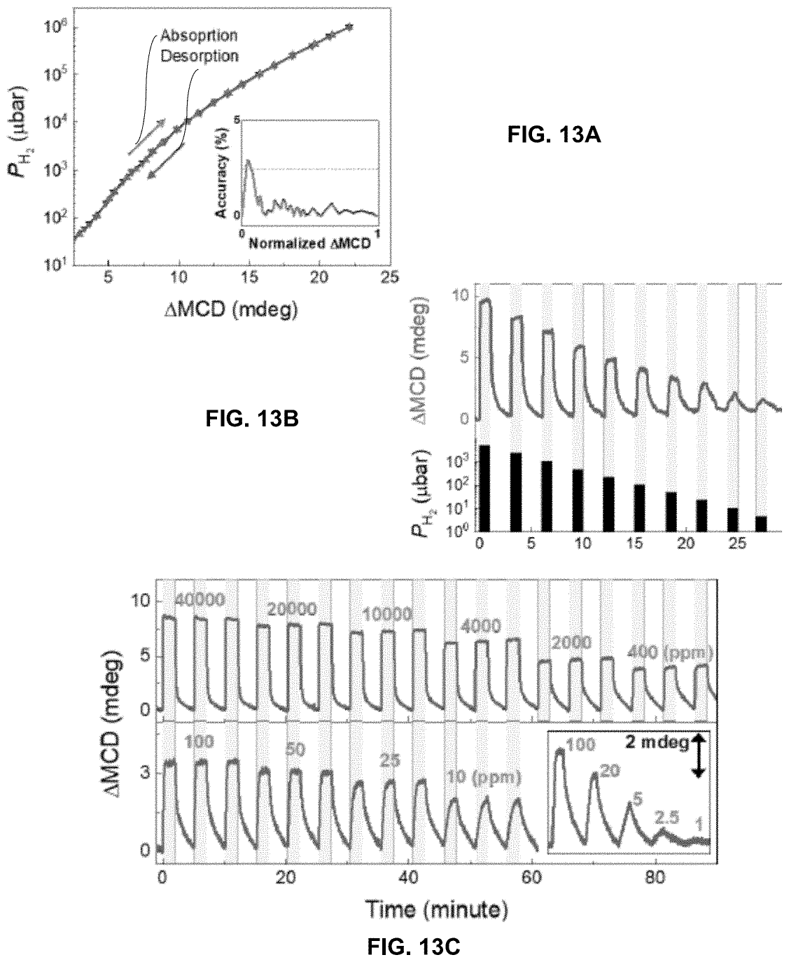

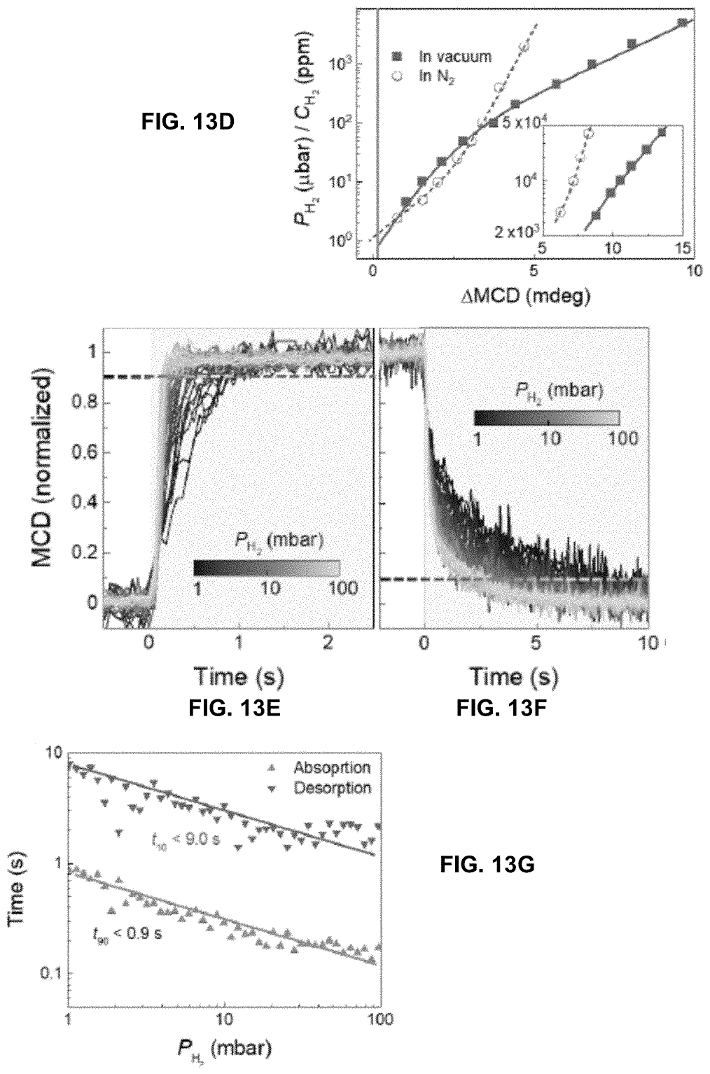

ΔMCD H₂ sorption isotherm of a sensor, where ΔMCD versus P_{H2} is extracted using a laser diode at λ=450 nm and B=+380 G, is shown in FIG. 13A. It was noted that the ΔMCD H₂ isotherm could be measured at any combination of λ and B. The shape of the ΔMCD isotherm curve was generally similar to that of an optical transmission isotherm, and no hysteresis was observed at the measured pressure range. In addition, the plateau pressure shifted significantly to higher pressure than that of a pure Pd sensor and was above the probing pressure range. The result was consistent with the previous observation for bulk PdCo alloys and similar to the behaviors of the PdCu alloy nanoparticles. As a result, the disclosed sensor could yield a very high sensor accuracy (<2.5% full-scale, and <1% when P_{H2}>1 mbar) and can be considered as “hysteresis-free” (FIG. 13A, inset).

The very high surface coverage (>90%) of the nanostructures with the sensing layer along with the unique utilization of MCD as the H₂ indicator allowed stable and sizable sensor responses at very low concentrations of H₂, which could hardly be resolved by using other optical-based readout technique such as transmission, extinction, or reflection with a PdCo sensor having a Co content up to >30 at. %. As demonstrated in FIG. 13A, the sensor showed distinct MCD responses to step-wise pressure pulse of pure H₂ from 5000 to 4.6 μbar at a 1 Hz sampling rate, and could clearly resolve the lowest H₂ pulse with a potential LOD at even lower P_{H2}. In the flow mode with N₂ as a gas carrier (flow rate of 400 ml/min at 1 atm), the sensor exhibited reproducible MCD responses with 3-cycles of each H₂ concentration (C_{H2}), ranging from 40000 ppm to 10 ppm (FIG. 13C). Further tests show that the sensor exhibited a distinct response from the background noise at C_{H2} as low as <2.5 ppm (FIG. 13C, inset). In order to quantitatively assess the LOD of the sensor, the noise evaluations were performed with different signal sampling frequency an experimental signal noise σ=0.04 mdeg at 1 Hz was obtained. Hence, by defining the LOD as 36=0.12 mdeg, the LOD was extrapolated to <1 μbar with pure H₂ and <1.5 ppm of H₂ in N₂ (FIG. 13D).

FIG. 13E presents raw absorption kinetic response of the sensor (with desorption pressure of <0.08 mbar) to varying H₂ pressure from 1000 to 1 mbar at 23° C. (32 Hz of sampling frequency). FIG. 13F presents raw desorption kinetic response of the sensor (with desorption pressure of <0.08 mbar) to varying H₂ pressure from 1000 to 1 mbar at 23° C. (32 Hz of sampling frequency). FIG. 13G presents extracted absorption time (t₉₀) and desorption time (t₁₀), derived from the data of FIG. 13E and FIG. 13F.

### Example 3

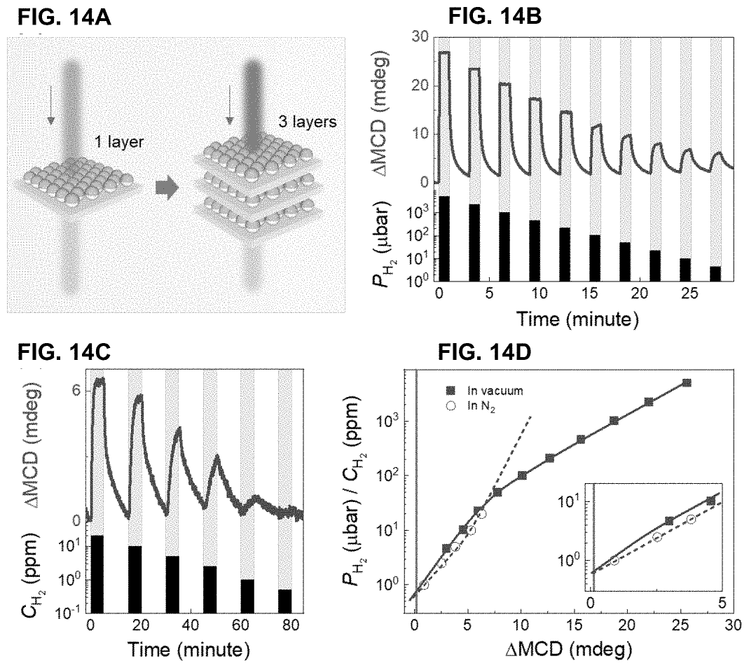

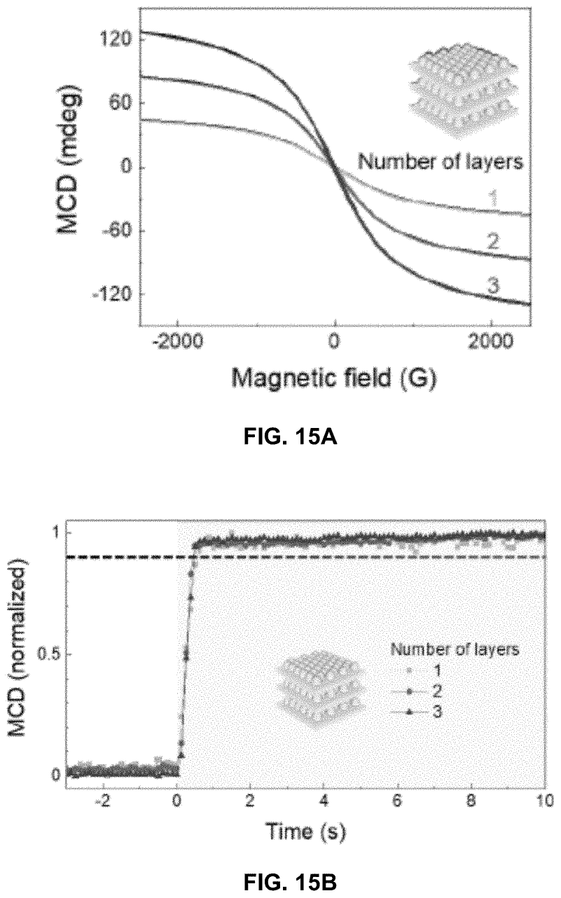

A representative triple-layer PdCo sensor (FIG. 14A) was examined with response time and LOD measurements as described above. As can be seen in FIG. 15A, the MCD signal of the stack is proportional to the number of stacking layers, regardless of the magnitude of B field. Expectedly a similar absorption kinetic was observed of 2- and 3-layer stacks with t₉₀<0.5 s at P_{H2}=10 mbar (FIG. 15B).

FIG. 14B shows the responses of the triple-layer sensor to multiple step-wise hydrogen pulses (an identical test with a single-layer was presented in FIG. 13B). The triple-layer sensor yielded an approximately 3-times larger MCD signal than that of the single-layer sensor (at a certain P_{H2}), and the lowest H₂ pulses of 4.6 μbar (the system limit) was clearly resolved. In addition, the triple-layer sensor could resolve a 1-ppm hydrogen pulse in nitrogen (FIG. 14C), which can hardly be seen with the single-layer system (FIG. 13C, inset). Consequently, the LOD was obtained to be <700 nbar and <700 ppb for pure H₂/vacuum and H₂ in nitrogen, respectively (FIG. 14D). It was expected that one could further lower the LOD by having more sensing layers, however, the sensor platform would require higher power consumption as a trade-off.

### Example 4

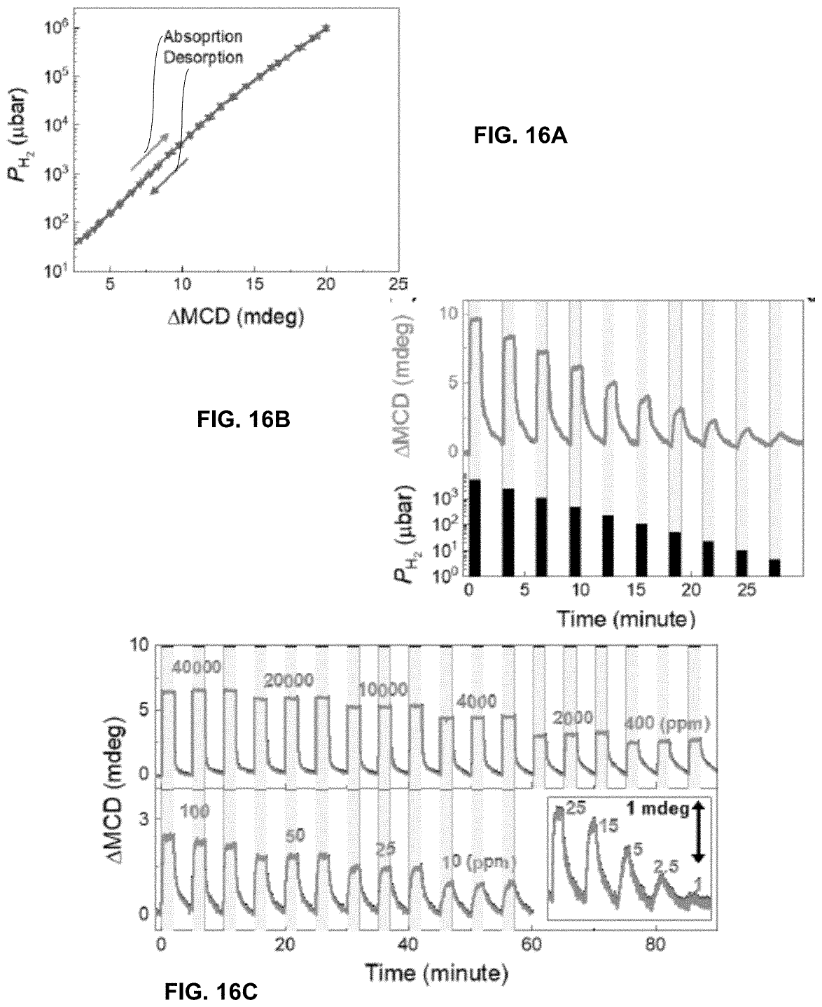

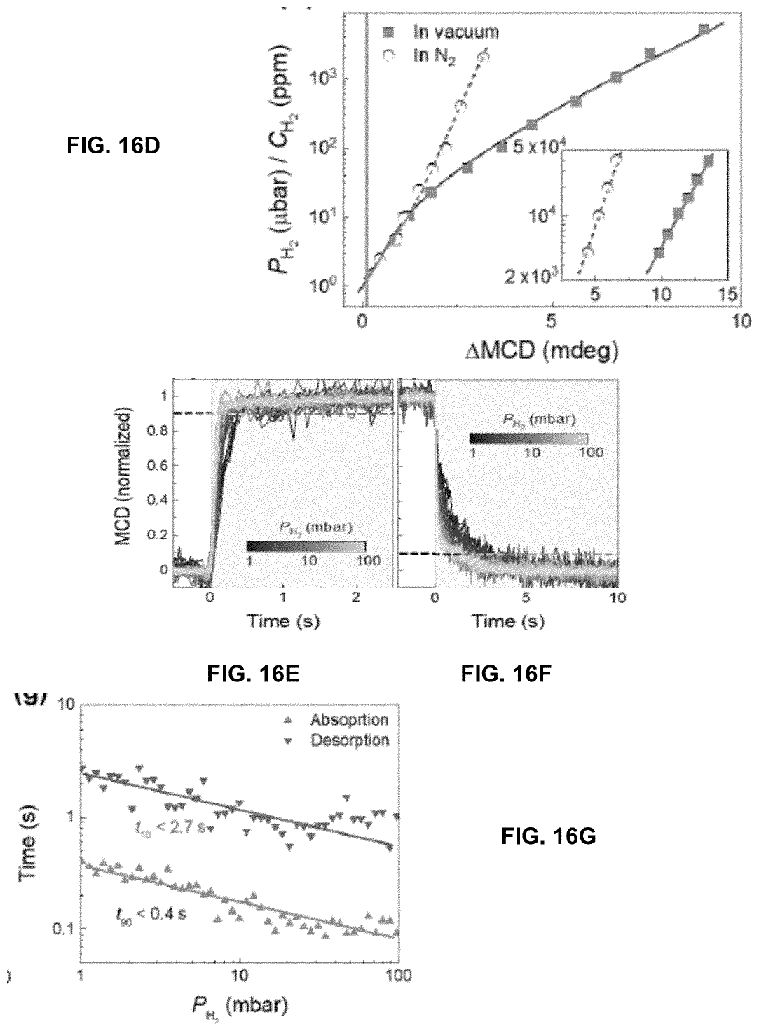

A 30-nm layer of TAF was coated on the sensing layer of nanoparticles of a sensor. The sensor was characterized in detail as shown in FIG. 16A-FIG. 16G. A similar MO behavior (MCD spectra, ΔMCD upon hydrogenation, and MCD magnetization) was seen in the TAF coated sensor as in the uncoated sensors. The extracted MCD sorption isotherm summarized in FIG. 16A shows a hysteresis-free characteristic and comparable signal level to an uncoated sensor. Further tests to examine the LOD of the sensor showed that it could resolve at as low as 4.6 μbar pulses of pure H₂ in vacuum (FIG. 16B) or 2.5 ppm of H₂ pulse in N₂ carrier gas (FIG. 16C). From the experimental results, it was extrapolated that the LOD was about 1 μbar with pure H₂ and <1.5 ppm of H₂ in N₂, which is on par with the uncoated sensor (FIG. 13D). Notably, the sorption times of the system were remarkably accelerated (FIGS. 16E and 16F). Three notable features are revealed (FIG. 16G): (1) the t₉₀ at P_{H2}=1 mbar was about 0.40 s, which is about 2 times faster than any optical hydrogen sensors under similar conditions; (2) the t₉₀ at P_{H2}=40 mbar (˜4 vol %) is <0.1 s, which is nearly twice as fast as any optical hydrogen sensors under similar conditions; and (3) the recovery times t₁₀ in the 1-100 mbar P_{H2} range are less than 2.7 s (2.7 s at 1 mbar and 1.0 s at 40 mbar (˜4 vol %)). Those record-low sorption times make this sensor one of the fastest sensors reported up to date under similar conditions. Along with the ppm-LOD, this sensing platform represents the current state-of-the-art in H₂ sensing.

### Example 5

As TAF is well-known for its high gas permeability but poor selectivity, it was expected that the sensor was not fully protected by the TAF coating layer toward the deactivation gases such as CO. To overcome this drawback, a layer of 100-nm of PMMA was coated on top of the TAF layer, as PMMA has been demonstrated as an excellent hydrogen selective membrane. Similar MCD and ΔMCD characteristics of the NP/TAF/PMMA sensor were seen as compared to uncoated sensors.

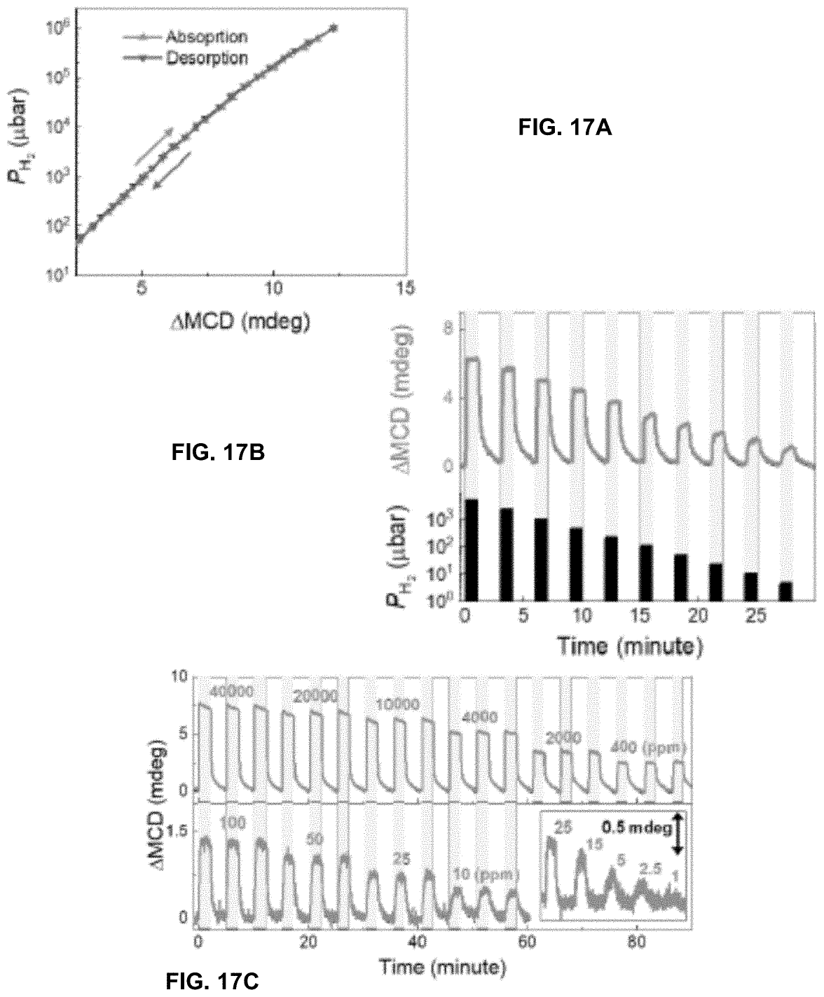

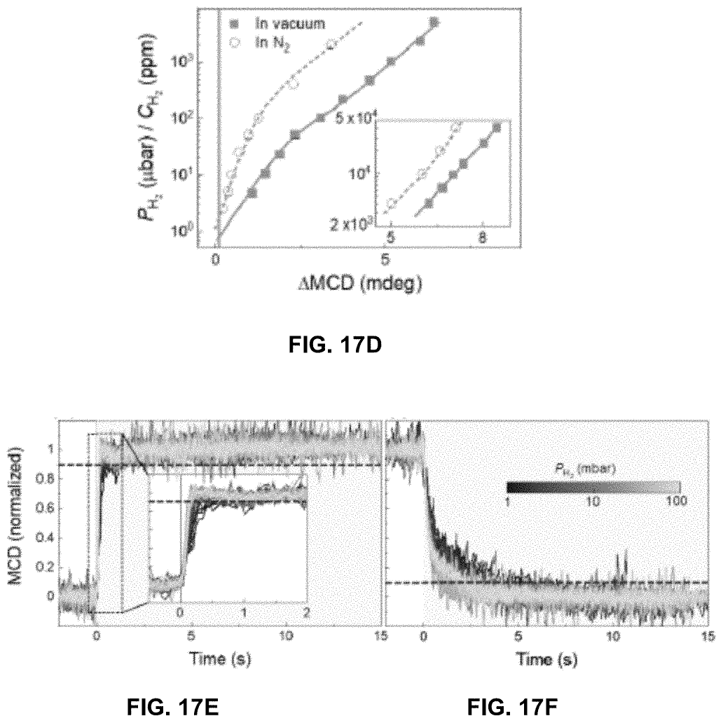

An identical sensing characterization was carried out for this sensor, which can be found in FIG. 17A-FIG. 17F. Remarkably, essentially no significant change in t₁₀ and t₉₀ of this sensor in comparison to the TAF coated (FIG. 16A) was observed, which implies that (i) coating PMMA did not block/slow-down H₂ sorption kinetics on the sensor and (ii) the ultrafast response time is ascribed to the reduced activation barrier induced at the TAF/PdCo interface. On the other hand, a notable performance which needs to be emphasized is the LOD of <0.9 μbar of pure H₂ in vacuum and ˜2 ppm of H₂ in N₂ (FIG. 17D).

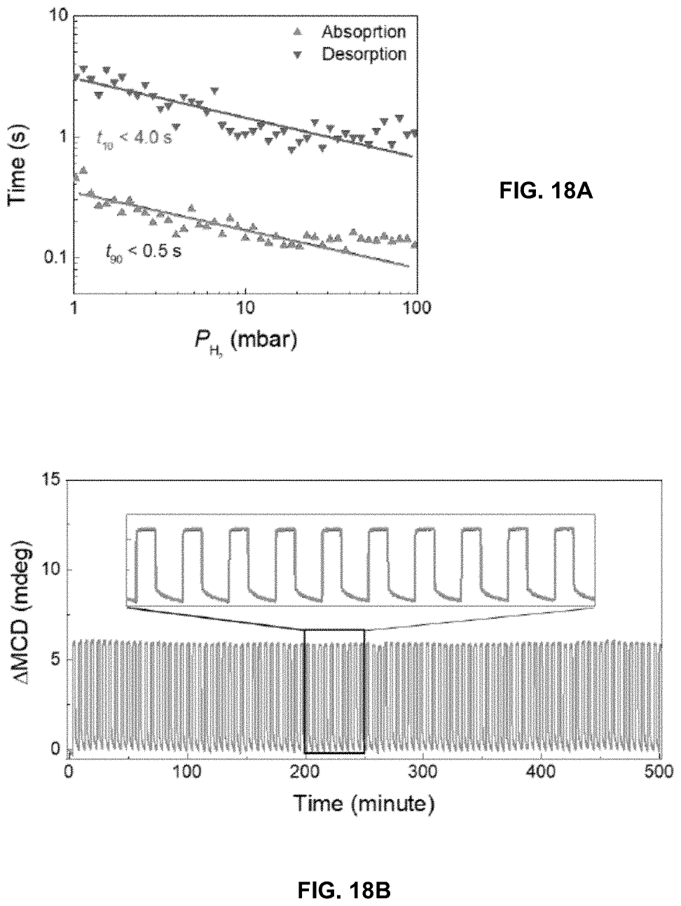

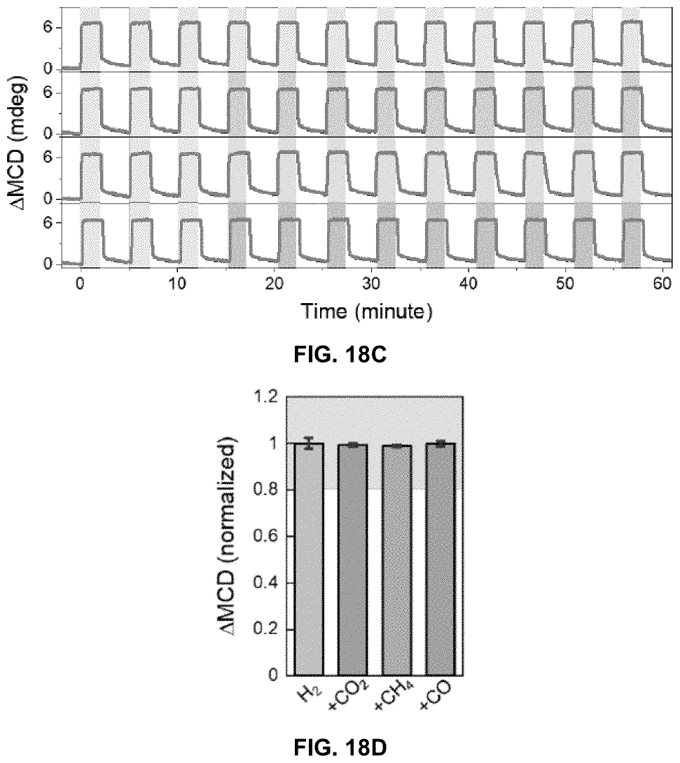

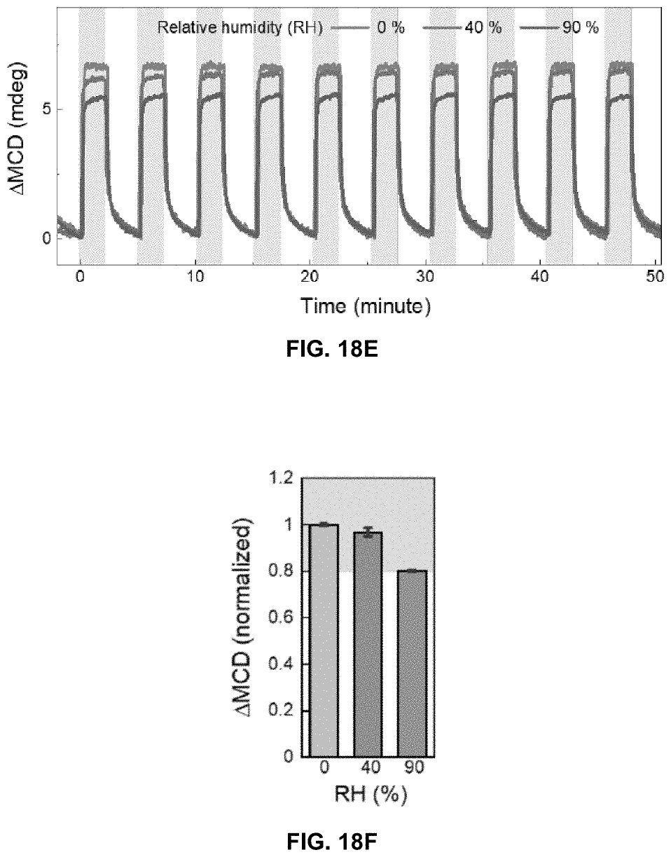

Further rigorous tests were carried out on the sensor to assess the operation in practical condition, such as sensing in a more realistic gas carrier like synthetic air, under the interferences from toxic gases (CO, CO₂, CH₄) and moisture. Results are shown in FIG. 18A-FIG. 18F. The sensor showed a great stability without the sign of degradation, upon >500 of (de)hydrogenation cycle (2% H₂ in synthetic air), and the first 100-cycles of a 5-month-old sample (stored in glove-box) are presented in FIG. 18B. In addition, an excellent selectivity of the sensor to high concentration pulses of CO (0.2%), CO₂ (5%), and CH₄ (5%) were observed (FIG. 18C, in which CO pulses are shown beginning at 15 min. on the second row, CO₂ pulses beginning at 15 min on the third row, and CH₄ pulses beginning at 15 min on the fourth row. No sign of signal degradation is witnessed upon 9 pulses of gas exposure (FIGS. 18C and 18D). It was noted that the CO₂ and CH₄ selectivity of the system might come from intrinsic resistances of PdCo materials, and may be enhanced by the selective PMMA coating. Furthermore, the sensor showed a great tolerance toward humidity (FIG. 18E), as the absolute response of the sensor remained within the ±20% deviation limit up to a high relative humidity (RH) of 90%. Additionally, by stacking this tandem sensor as presented above, it is possible to realize an exemplary optical hydrogen sensor platform with sub-0.5-second response, ppb-LOD, and robustness against interfering gases and moistures.

The Pd₆₇Co₃₃/TAF/PMMA MCD sensor exhibited the response time of <0.5 s and recovery time of <4.0 s from 1-100 mbar of H₂ partial pressure. The sensor preserved excellent accuracy (<2.5% full scale), <1 ppm limit of detection (LOD), strong selectivity against interference gases against interference gases such as moisture, O₂, CO, CO₂ and CH₄, and slow aging effect. The MCD H₂ sensor outperformed the state-of-the-art optical sensors reported to date and potentially satisfies the most challenging performance targets imposed by DOE.

Overall, disclosed sensors and systems using disclosed signal readout technique demonstrate a viable path forward to spark-proof optical sensors for H₂ detection applications. The MCD sensors could have a great impact on developing other high performance optical gas sensors in general. In addition, understanding the interaction between hydrogen and magnetic hydrides could boost the development of fast proton-based magneto-ionic devices for not only spintronic applications but also for superior active gas sensors.

While certain embodiments of the disclosed subject matter have been described using specific terms, such description is for illustrative purposes only, and it is to be understood that changes and variations may be made without departing from the spirit or scope of the subject matter.

What is claimed is:

1. A hydrogen sensor comprising a substrate and a sensing layer on a surface of the substrate, the sensing layer comprising a composite that includes a hydride-forming material and a magnetic material, the composite including the magnetic material and the hydride-forming material in a molar ratio of from about 1:1 to about 1:6, the sensing layer having a thickness of from about 1 nanometers to about 30 nanometers.

2. The hydrogen sensor of claim 1, wherein the hydride forming material comprises palladium, platinum, magnesium, titanium, vanadium, or any combination thereof.

3. The hydrogen sensor of claim 1, wherein the magnetic material comprises cobalt, iron, nickel, gadolinium, or any combination thereof.

4. The hydrogen sensor of claim 1, wherein the sensing layer forms a continuous coating on about 5% or more of the surface of the substrate.

5. The hydrogen sensor of claim 1, wherein the surface is non-planar.

5. The hydrogen sensor of claim 1, further comprising one or more additional layers on the surface of the substrate.

6. The hydrogen sensor of claim 5, at least one of the one or more additional layers covering the sensing layer.

7. The hydrogen sensor of claim 6, the at least one layer comprising a polytetrafluoroethylene, a polymethyl-methacrylate, a polyimide, a polysulfone, a siloxane, or any combination thereof.

8. The hydrogen sensor of claim 1, further comprising a noble metal.

9. The hydrogen sensor of claim 8, wherein the noble metal is a component of the composite.

10. The hydrogen sensor of claim 8, the sensor further comprising a layer adjacent the sensing layer, the adjacent layer comprising the noble metal.

11. The hydrogen sensor of claim 1, the substrate comprising a particle.

12. The hydrogen sensor of claim 11, the hydrogen sensor comprising an array of the particles.

13. The hydrogen sensor of claim 1, the substrate comprising a nano-hole array.

14. The hydrogen sensor of claim 1, the hydrogen sensor comprising a first layer comprising the substrate and the sensing layer on the surface of the substrate and further comprising one or more additional layers in stacked arrangement with the first layer, each of the one or more additional layers comprising additional substrates and a sensing layer on a surface of each of the additional substrates.

15. A hydrogen sensing system comprising:
a hydrogen sensor comprising a substrate and a sensing layer on a surface of the substrate, the sensing layer comprising a composite that includes a hydride-forming material and a magnetic material, the composite including the magnetic material and the hydride-forming material in a molar ratio of from about 1:1 to about 1:6, the sensing layer having a thickness of from about 1 nanometers to about 30 nanometers;
a source configured to contact the sensor with a probing energy beam;
a magnet, wherein the hydrogen sensor is retained within a magnetic field of the magnet; and
an analysis system configured to detect and analyze a resulting energy beam resulting from interaction of the hydrogen sensor and the probing energy beam.

16. The system of claim 15, further comprising one or more optical modulation devices configured to interact with the probing energy beam or the resulting energy beam.

17. The system of claim 16, the one or more optical modulation devices comprising a polarizer, a phase sensitive photo-elastic modulator, a quarter wave-plate, an analyzer, or any combination thereof.

18. A method for detecting hydrogen comprising:
contacting a sensor retained within a magnetic field with a sample comprising hydrogen, the sensor comprising a substrate and a sensing layer on a surface of the substrate, the sensing layer comprising a composite that includes a hydride-forming material and a magnetic material, the composite including the magnetic material and the hydride-forming material in a molar ratio of from about 1:1 to about 1:6, the sensing layer having a thickness of from about 1 nanometers to about 30 nanometers;
contacting the sensor with a probing energy beam; and
analyzing the magneto-optic response of the probing energy beam following the contact with the sample and the sensor, the analysis providing information regarding the presence or quantity of the hydrogen in the sample.

19. The method of claim 18, the magneto-optical response analysis comprising Faraday rotation analysis, magneto-optical Kerr rotation analysis, reflective/transmission magnetic-circular dichroism analysis, or any combination thereof.

20. The method of claim 18, wherein the sensor exhibits a response time to the sample of 1 second or less at room temperature across a hydrogen concentration range from 0.1% to 10% by volume of the sample.
